(19) United States
(12) Patent Application Publication
(10) Pub. No.: US 2011/0022177 A1
(43) Pub. Date: Jan. 27, 2011

(54) INTERVERTEBRAL FILLING FOR CERVICAL VERTEBRAE

(75) Inventor: Chung-Chun YEH, Taipei (TW)

Correspondence Address: BACON & THOMAS, PLLC, 625 SLATERS LANE, FOURTH FLOOR, ALEXANDRIA, VA, 22314-1176, US

(73) Assignee: PAONAN BIOTECH CO., LTD., Taipei (TW)

(21) Appl. No.: 12/854,934

(22) Filed: Aug. 12, 2010

Related U.S. Application Data
(63) Continuation-in-part of application No. 11/360,383, filed on Feb. 24, 2006, now abandoned.

(51) Int. Cl. A61F 2/44

(52) U.S. Cl. 623/17.15

(57) ABSTRACT

An intervertebral filling for cervical vertebrae end-plates includes a supporting body, and a buffer layer surrounding the supporting body. The supporting body has a Shore A hardness of 50-80 and the buffer layer has a Shore A hardness of 20-45. The supporting body has a height greater than that of the buffer layer, so that the supporting body protrudes about 1 mm to 3 mm from the buffer layer at two opposite ends thereof.

### CROSS-REFERENCE TO RELATED APPLICATION

The present application is a continuation-in-part application of U.S. patent application Ser. No. 11/360,383, filed Feb. 24, 2006, the entire contents of which are incorporated herein by reference.

### BACKGROUND OF THE INVENTION

1. Field of the Invention

The present invention relates to an intervertebral filling adapted for placement between two end-plates of two adjacent vertebrae in the human body, and in particular to an intervertebral filling adapted for placement between two end-plates of two adjacent cervical vertebrae in the human body.

2. Description of the Related Art

Human intervertebral discs between spinal vertebrae end-plates have an interesting mechanical structure. The intervertebral disc has a nucleus propulsus and an annulus fibrosus. The nucleus propulsus has a diameter of about 1.5 cm and is composed mainly of liquid. The annulus fibrosus has multiple layers and stabilizes the core. The intervertebral disc has the nature of a buffer for the human body, and helps to prevent damage to the spine.

Due to various causes, such as moving heavy loads, inappropriate exercises, incorrect posture over long periods of time, pregnancies or back injuries, the spine in the human body may suffer herniated intervertebral disc problems; minor conditions may simply cause enlargement of the annulus fibrosus, but serious conditions can cause the annulus fibrosus to break and the nucleus propulsus to leak, thereby causing compression of the vertebral nerves, which is termed herniation of the intervertebral disc. This condition is very common among the elderly, and a medical solution involves performing an operation to install an artificial intervertebral disc. The following describes the structure of a prior art artificial intervertebral disc.

Please refer to FIG. 1 and FIG. 2. FIG. 1 is a schematic drawing of a prior art artificial disc. FIG. 2 is a schematic drawing of the prior art artificial disc installed on spinal vertebrae. The prior art artificial intervertebral disc comprises a main body 10. The main body 10 has a plurality of grooves 100. A fillister 110 is formed on a spinal vertebra end-plate 11 matching the shape of the main body 10. When the main body 10 is fixed in the fillister 110, a plurality of grooves 100 need to be formed on the spinal vertebra end-plate 11 for newly grown bone, which increases the difficulty of the surgical operation. Moreover, the prior art artificial intervertebral disc is made of metal and rubs against the spinal vertebra end-plate 11 when the patient moves, which cause discomfort and deterioration of the spinal vertebra end-plate 11.

To improve comfort, U.S. Pat. No. 6,022,376, No. 5,824,093 and No. 5,674,295 disclose an artificial intervertebral disc having a main body 12 made of hydrogel and covered by a jacket 13 made of polyethylene. Please refer to FIG. 3 and FIG. 4. FIG. 3 is a schematic drawing of another prior art artificial disc. FIG. 4 is a schematic drawing of another prior art artificial disc installed on spinal vertebrae. Two ends of the jacket 13 have a plurality of fastening members 14. A spinal vertebra end-plate 15 has a plurality of corresponding holes (not shown) so the plurality of fastening members can be fastened in the holes on the spinal vertebra end-plate 15 to complete the artificial disc. Since this artificial disc is mainly made of hydrogel, it provides good elasticity and reduces stress on the spine. However, due to softness, the fastening member 14 does not provide a good locking effect, which can cause exposure and displacement of the artificial disc.

Therefore, it is desirable to provide an artificial intervertebral disc to mitigate and/or obviate the aforementioned problems.

### SUMMARY OF THE INVENTION

A main objective of the present invention is to provide a comfortable and stable intervertebral filling, which is adapted for placement between two end-plates of two adjacent spinal vertebrae in the human body, and in particular for placement between two end-plates of two adjacent cervical vertebrae in the human body.

The intervertebral filling of the present invention comprises a first surface and a second surface wherein the intervertebral filling is capable of being placed between two cervical vertebrae end-plates so that the first surface and the second surface of the intervertebral filling contact the two cervical vertebrae end-plates and provide support between the two cervical vertebrae end-plates; a supporting body having an upper surface, a lower surface and a longitudinal surface extending from the upper surface to the lower surface in a continuous manner, wherein the supporting body is positioned in the intervertebral filling in an axial position so that the upper surface of the supporting body is a part of the first surface of the intervertebral filling and the lower surface of the supporting body is a part of the second surface of the intervertebral filling; and a buffer layer which surrounds and is contiguous with the longitudinal surface of the supporting body. The intervertebral filling also optionally includes at least two positioning means. Each positioning means protrudes from the supporting body oppositely so that it can be mounted on a cervical vertebra end-plate to provide a positioning effect.

The supporting body of the intervertebral filling of the present invention comprises a cylindrical support part and two caps connected to an upper end and a lower end of the cylindrical support separately, wherein the two caps are adapted to contact the two cervical vertebrae end-plates separately. The buffer layer of the present invention surrounds and is contiguous with the cylindrical support part so that the two caps protrudes from the buffer layer with a height of about 1 mm to about 3 mm, respectively, whereby the two caps can be received in two corresponding recesses formed on the two cervical vertebrae end-plates respectively, while the buffer layer is conformably pressed by intact portions on the two cervical vertebrae end-plates, wherein the cylindrical support part has a Shore A hardness of 50-80 and the buffer layer has a Shore A hardness of 20-45, and the cylindrical support part and the buffer layer are independently made of pharmaceutically acceptable polymeric materials.

Preferably, the cylindrical support part and the buffer layer are independently made of silicone rubber or polyurethane elastomer.

Preferably, the two caps are porous metallic caps.

Preferably, each of the two caps has a post, and the cylindrical support part has a hole on the upper end and another hole on the lower end thereof corresponding to the posts, and the two caps are connected to the upper end and the lower end of the cylindrical support part separately with the posts of the two caps being received in said hole and said another hole.

Preferably, the two caps of the supporting body form the upper surface and the lower surface of the supporting body, and the cylindrical support part forms the longitudinal surface of the supporting body.

The buffer layer of the present invention is made of an elastic material so that the buffer layer does not cause inconvenience during movement of the patient, and does not suffer any wear or damage. The cylindrical supporting part is a harder than the buffer layer and is used for sharing stress in the spine, providing a strong support. The sharp ends of the optional positioning means can be inserted into the surface of the adjacent cervical vertebrae end-plates. Consequently, the intervertebral filling of the present invention remains stably positioned between the two adjacent cervical vertebrae end-plates without undergoing displacement.

Other objects, advantages, and novel features of the invention will become more apparent from the following detailed description when taken in conjunction with the accompanying drawings.

### BRIEF DESCRIPTION OF THE DRAWINGS

FIG. 1 is a schematic drawing of a prior art artificial intervertebral disc.

FIG. 2 is a schematic drawing of the prior art artificial intervertebral disc installed on spinal vertebrae.

FIG. 3 is a schematic drawing of another prior art artificial intervertebral disc.

FIG. 4 is a schematic drawing of another prior art artificial intervertebral disc installed on spinal vertebrae.

FIG. 5 illustrates an intervertebral filling constructed according to a first preferred embodiment of the present invention.

FIG. 6 is a cross-sectional view of the intervertebral filling shown in FIG. 5.

FIG. 7 is a cross-sectional view of an intervertebral filling constructed according to a second preferred embodiment of the present invention.

FIG. 8 is a schematic drawing showing the intervertebral filling shown in FIG. 5 implanted between two spinal vertebrae end-plates in a human body.

FIG. 9 is a schematic drawing showing the intervertebral filling shown in FIG. 5 implanted between two spinal vertebrae end-plates under compression.

FIG. 10 illustrates an intervertebral filling constructed according to a third preferred embodiment of the present invention.

FIG. 11 illustrates an intervertebral filling constructed according to a fourth preferred embodiment of the present invention.

FIG. 12 is a cross-sectional view of the intervertebral filling shown in FIG. 11.

FIG. 13 is a schematic drawing showing an intervertebral filling constructed according to a fifth preferred embodiment of the present invention before being assembled.

FIG. 14 is a schematic drawing showing the intervertebral filling shown in FIG. 13 after being assembled.

FIG. 15 is a schematic drawing showing the intervertebral filling shown in FIG. 14 implanted between two spinal vertebrae end-plates.

FIG. 16 is a schematic drawing showing an intervertebral filling constructed according to a sixth preferred embodiment of the present invention.

FIG. 17 is a schematic drawing showing an intervertebral filling constructed according to a seventh preferred embodiment of the present invention.

FIG. 18 is a schematic drawing showing an intervertebral filling constructed according to an eighth preferred embodiment of the present invention.

FIG. 19 is a schematic drawing showing an intervertebral filling constructed according to a ninth preferred embodiment of the present invention.

FIG. 20a is a schematic perspective view showing an intervertebral filling before being assembled, which is constructed according to a tenth preferred embodiment of the present invention.

FIG. 20b is a schematic side plan view showing the intervertebral filling of the tenth preferred embodiment of the present invention in use.

FIG. 21 is a schematic top view showing the intervertebral filling of the tenth preferred embodiment of the present invention.

FIG. 22 is a schematic side view showing the intervertebral filling of the tenth preferred embodiment of the present invention.

FIG. 23 is a plot showing the Shore A hardness of various polymeric materials.

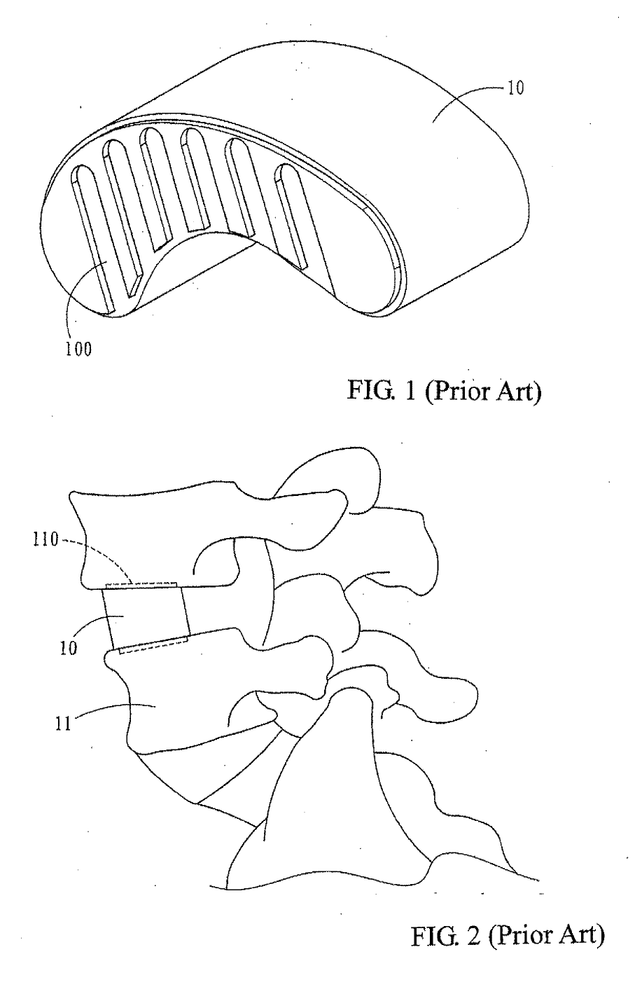

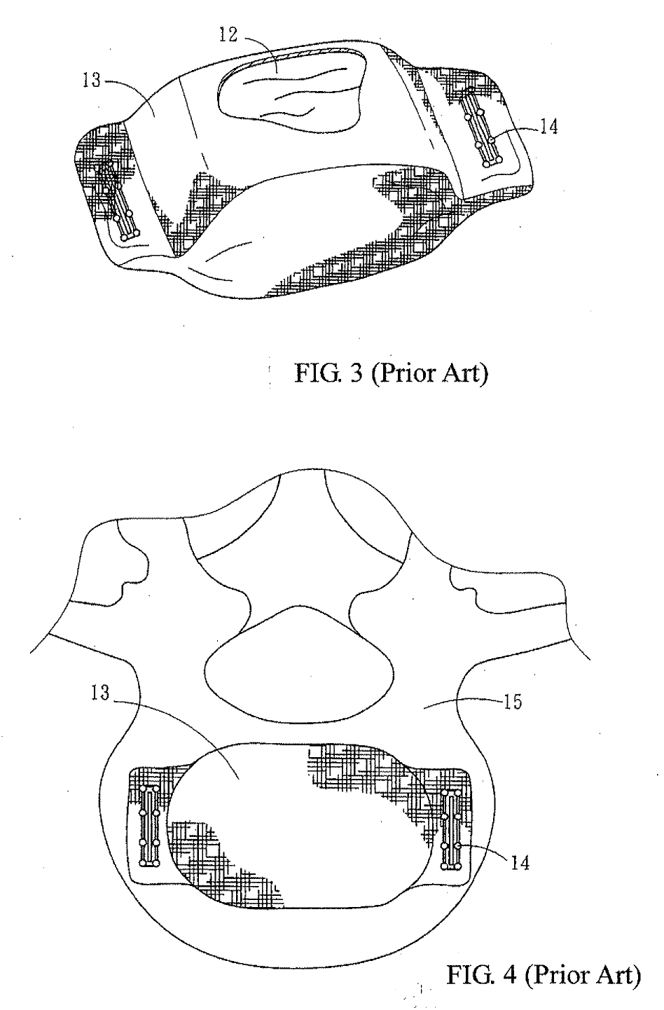

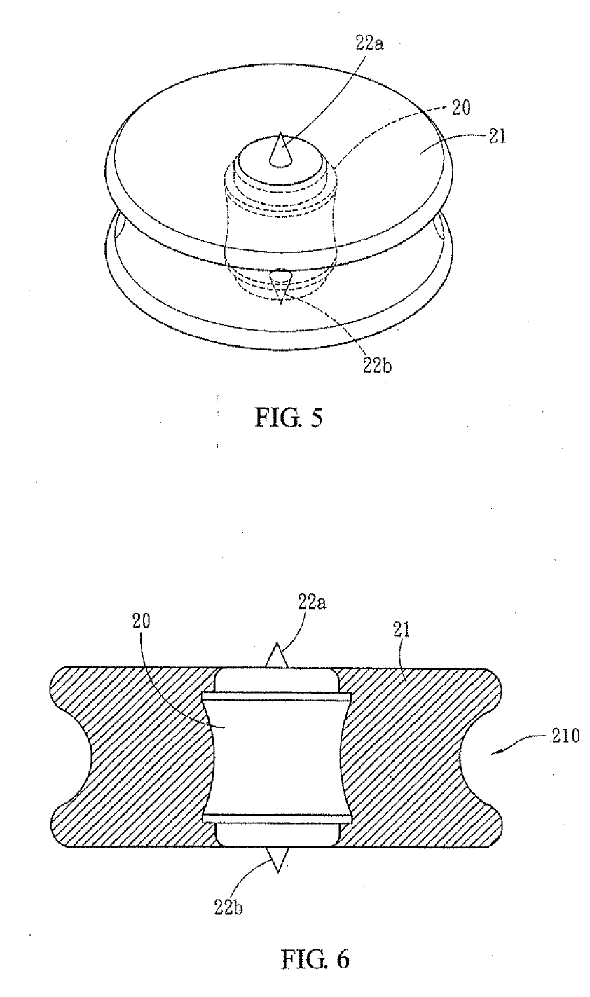

### DETAILED DESCRIPTION OF THE PREFERRED EMBODIMENTS

The present invention provides an intervertebral filling adapted for placement between any two adjacent spinal vertebrae end-plates in the human body, which has a simple design and that can be firmly mounted in the human body. The following description, in conjunction with the accompanying drawing, explains the present invention in detail.

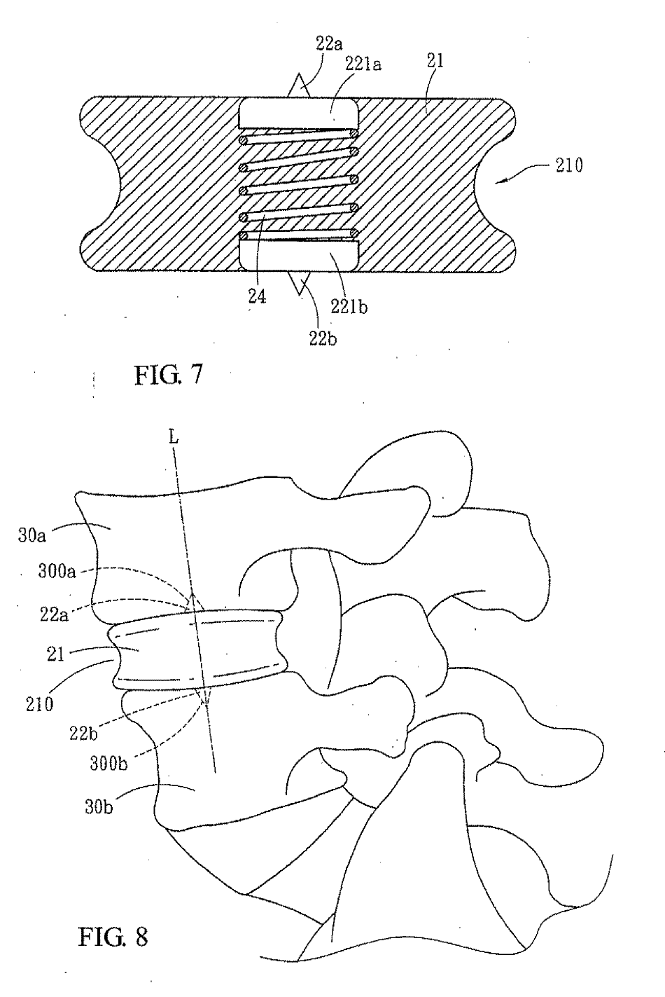

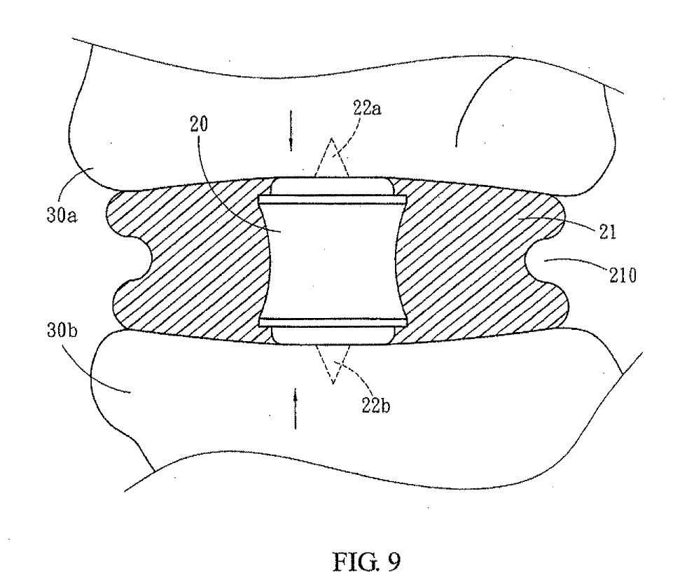

Please refer to FIG. 5, FIG. 6 and FIG. 8. FIG. 5 illustrates an intervertebral filling according to a first preferred embodiment of the present invention. FIG. 6 is a cross-sectional view of the intervertebral filling shown in FIG. 5. As shown in the drawings, the intervertebral filling of the present invention can be placed between any two adjacent spinal vertebrae end-plates 30a, 30b in the human body. During a surgical operation, the spinal vertebrae end-plates 30a, 30b can be held open with suitable tools, and the intervertebral filling can be placed between the spinal vertebrae end-plates 30a, 30b by tools that gently press upon upper and lower surfaces of the intervertebral filling. Subsequently, the tools separating the spinal vertebra end-plates 30a, 30b can be removed so that the spinal vertebrae end-plates 30a, 30b align with the intervertebral filling. A sharp end of two positioning pins 22a, 22b is respectively inserted into the surfaces of the spinal vertebra end-plates 30a, 30b to stabilize the spinal vertebra filling. The spinal vertebrae end-plates 30a, 30b may separately have at least one hole 300a, 300b (as shown in FIG. 8) on their surfaces for placement of the positioning pins 22a, 22b of the intervertebral filling. Additionally, a groove may be formed on the spinal vertebrae end-plates 30a, 30b to allow the entrance of the positioning pins 22a, 22b of the intervertebral filling, so that the entire intervertebral filling can slide into the spine. However, such operations are a very well known technology involving many different methods, and no further description is required.

The intervertebral filling comprises a buffer layer 21, placed between the two adjacent spinal vertebrae end-plates 30a, 30b and providing a buffering effect between the two spinal vertebrae end-plates 30a, 30b. This buffer layer 21 can be made of a soft plastic material or a hydrogel material that is flexible so that the buffer layer 21 does not cause any inconvenience during movement of the patient, and does not suffer any wear or damage. In this preferred embodiment, a concave groove 210 recessed perpendicularly to the direction of a center axis L of the supporting body (as shown in FIG. 8, and in the same direction as the spine) is formed on the peripheral surface of the buffer layer 21 to form a buffer area. With the groove 210, when the two spinal vertebrae end-plates 30a, 30b are pressed towards each other due to pressure from the spine, the buffer layer 21 will not be pushed out of the spine. The thickness of the buffer layer 20 may have various designs according to the positions of the two spinal vertebrae end-plates 30a, 30b to provide suitable ergonomics and curve angles for the two spinal vertebrae end-plates 30a, 30b.

The intervertebral filling further comprises a supporting body 20, which is covered by the buffer layer 21. Preferably, the supporting body 20 is made of an elastic plastic material, but should be harder than the material of the buffer layer 21 to provide support between the two spinal vertebrae end-plates 30a, 30b.

The intervertebral filling also comprises at least two positioning pins 22a, 22b. Ends of the positioning pins 22a, 22b are fixed on the two axial ends of the supporting body 20, and two sharp ends of the positioning pins 22a, 22b protrude from the buffer layer 21 and may be inserted into two pre-formed holes 300a, 300b on the two spinal vertebrae end-plates 30a, 30b. The two positioning pins 22a, 22b stabilize the supporting body 20 and the buffer layer 21 within the proper position between the two spinal vertebrae end-plates 30a, 30b. Preferably, the two positioning pins 22a, 22b can be made of metal or a hard material (such as plastic) so as to be able to stick into the surfaces of the two spinal vertebrae end-plates 30a, 30b and provide support along the direction of the spine.

In a second preferred embodiment, the supporting body 20 shown in FIG. 5 may have a different design. Please refer to FIG. 7. An elastic structure 24 is placed between the two positioning pins 22a, 22b in this embodiment, and the bases 221a, 221b of the two positioning pins 22a, 22b and the elastic structure 24 are all covered by the buffer layer 21. The elastic structure 24 provides support in the buffer layer 21 so that the two positioning pins 22a, 22b can also provide support along the axial direction; the elastic structure 24 is a compression spring, and can be of various types, such as spiral spring or disc spring. Additionally, the elastic structure 24 may be made of a metal or plastic material. This design can be an alternate for the supporting body 20 shown FIG. 5.

With the above-mentioned structure, the present invention is stable between the two spinal vertebrae end-plates 30a, 30b and forms an artificial spinal disc. With the buffer layer 21, the patient will feel more comfortable and have no directional limitations. The supporting body 20 with elastic material or the elastic structure 24 provides support, and the sharp ends of the two positioning pins 22a, 22b may be inserted into the holes 300a, 300b opened in the surface of the two spinal vertebrae end-plates 30a, 30b. Therefore, when the present invention is mounted in the patient, it remains securely positioned when the patient moves, without dislocating. The intervertebral filling of the present invention is made of an elastic material, and so is easily placed in the patient in a surgical procedure that requires only a small incision. The best placement position may be in the neck vertebrae or any other vertebrae within the human spine, and therefore the positional placement of the invention should not be limited to particular regions within the spine.

Please refer to FIG. 9. FIG. 9 is a schematic drawing showing the present invention being compressed. As shown, the buffer layer 21 on the peripheral surface thereof has a groove 210 recessed toward the axial center L (as shown in FIG. 8, this is along the direction of the spine) of the supporting body 20 to form a buffer area, which ensures the buffer layer 21 is not squeezed out from between the two spinal vertebrae end-plates 30a, 30b when the intervertebral filling suffers an external axial force.

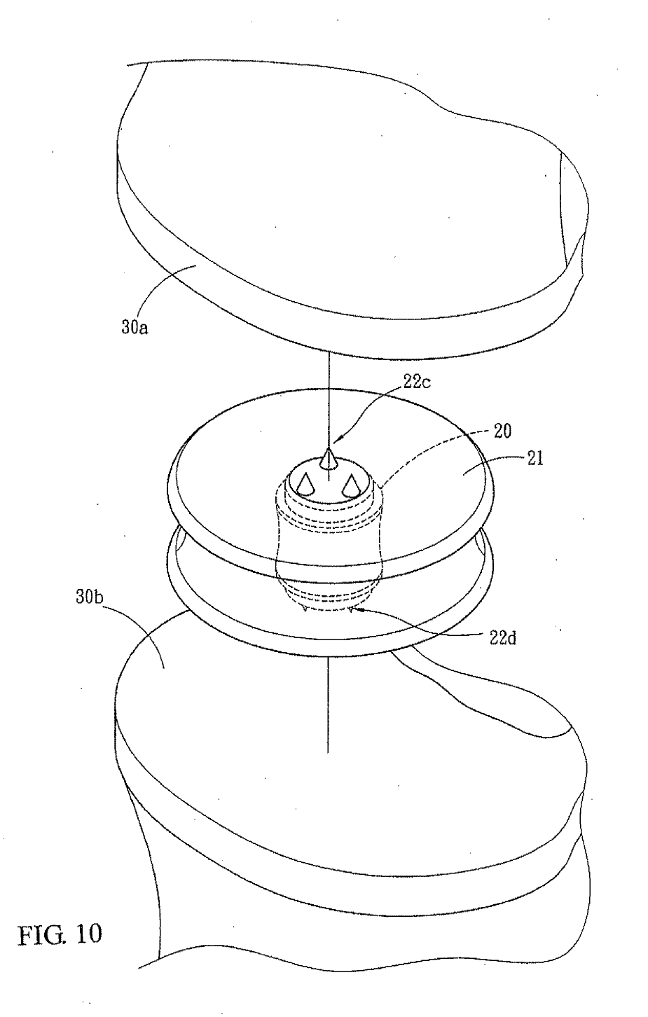

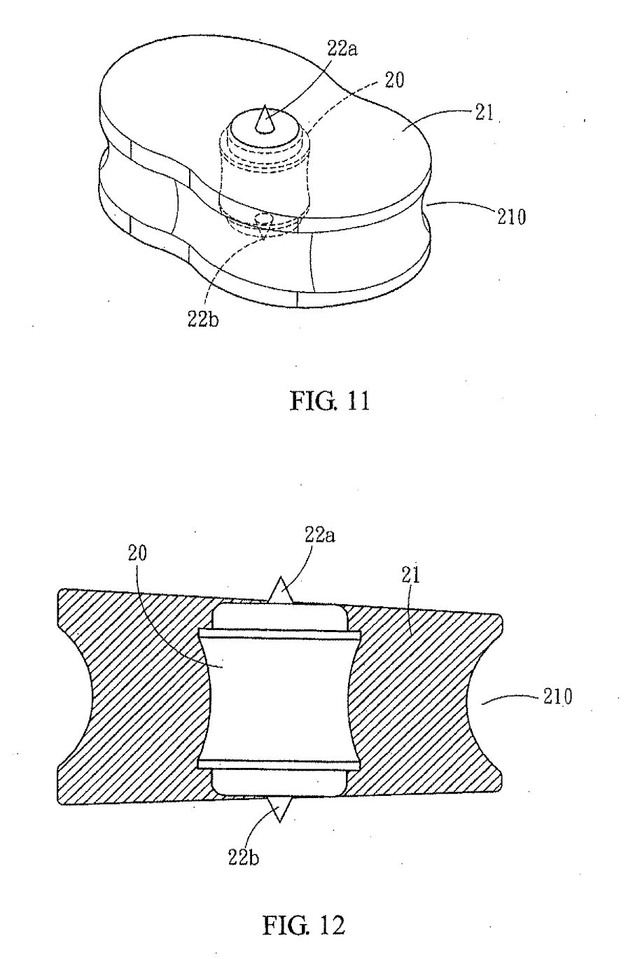

Please refer to FIG. 10. FIG. 10 illustrates a third preferred embodiment of the present invention. As shown in the drawing, there are three positioning pins 22c, 22d, 22e on the supporting body 20, which can provide more stability to the entire intervertebral filling.

Please refer to FIG. 11 and FIG. 12. FIG. 11 illustrates a fourth preferred embodiment of the present invention. FIG. 12 is a cross-sectional drawing of the embodiment of the present invention shown in FIG. 11. In this embodiment of the present invention, the buffer layer 21 can be designed to have a shape (as indicated by the kidney shape shown in the drawing) conformal with the shape of the two spinal vertebrae end-plates 30a, 30b, or the thickness of the buffer layer 21 can be varied to satisfy the different curvature requirements of the human spine.

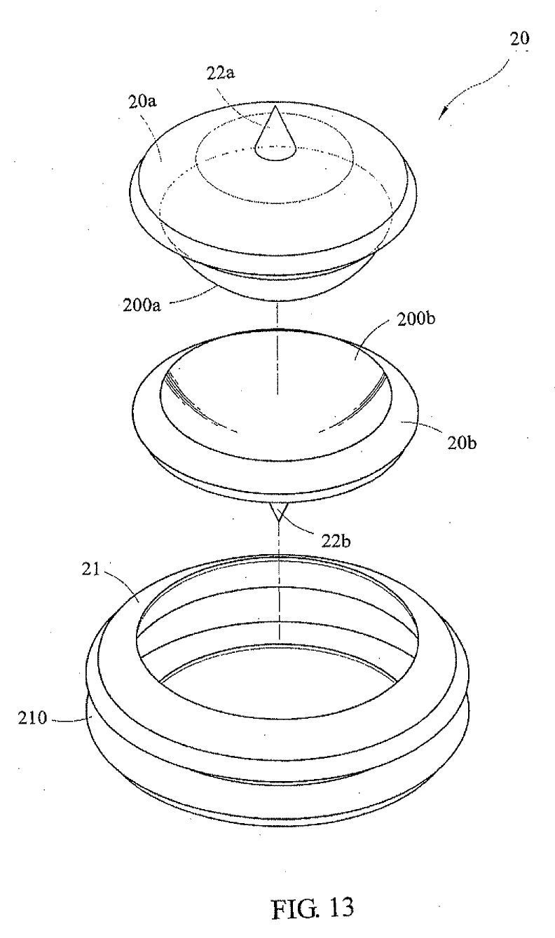

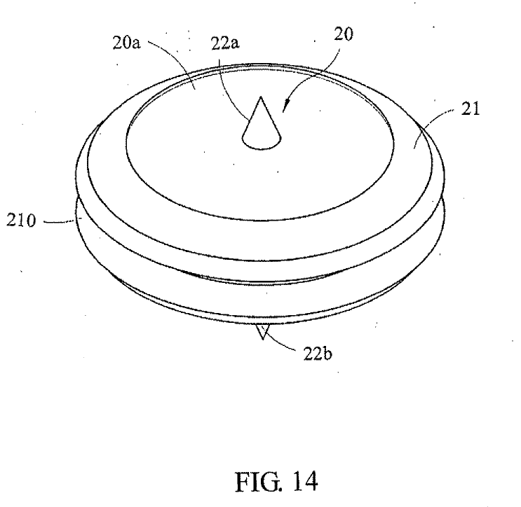

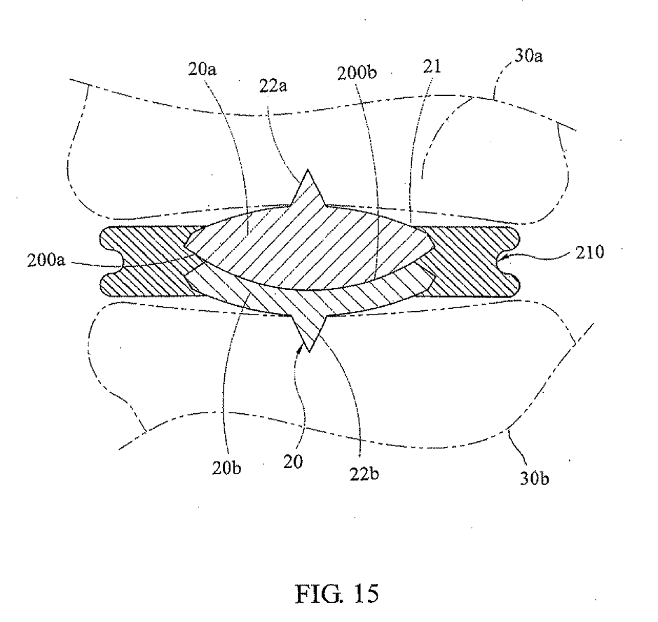

A fifth preferred embodiment is shown in FIGS. 13, 14 and 15, wherein the supporting body 20 is formed of a lower concave-up disk 20b having an upper concave-up surface 200b, and an upper eye-shaped part 20a having a lower spherical surface 200a, wherein the lower spherical surface 200a is received on the upper concave-up surface 200b to form a universal joint between the upper eye-shaped part 20a and lower concave-up disk 20b. The supporting body 20 is embedded in an elastic buffer layer 21, so that the upper eye-shaped part 20a and lower concave-up disk 20b are relatively movable to each other and the relative movement is confined by the elasticity of the buffer layer 21. Two positioning pins 22a, 22b are formed on the two axial ends of the supporting body 20 with two sharp ends of the positioning pins 22a, 22b protruding from the buffer layer 21 which are to be inserted into the two spinal vertebrae end-plates 30a, 30b. The buffer layer 21 has a groove 210 on the peripheral surface thereof to form a buffer area, which ensures the buffer layer 21 is not squeezed out from between the two spinal vertebrae end-plates 30a, 30b when the intervertebral filling suffers an external axial force. The materials of making the supporting body 20, positioning pins 22a, 22b, and buffer layer 21 are same as those described in the above preferred embodiments.

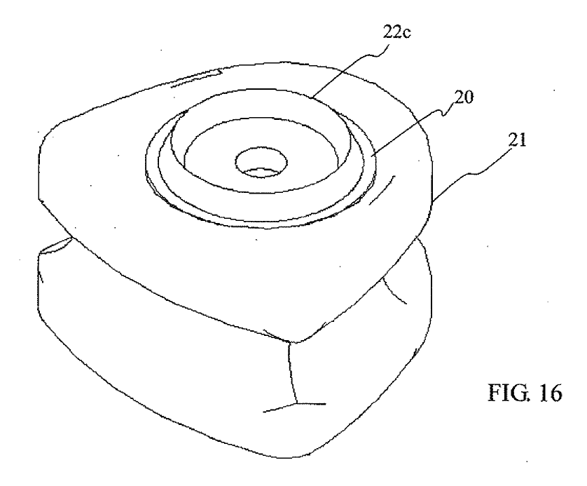

An intervertebral filling constructed according to a sixth preferred embodiment of the present invention is shown in FIG. 16, which has a construction similar to the first embodiments shown in FIGS. 5 and 6 except a major difference that a pair of position ring 22c having a sharp ridge are provided on the two axial ends of the supporting body 20 with the sharp ridge of the positioning ring 22c protruding from the buffer layer 21 to replace the position pins.

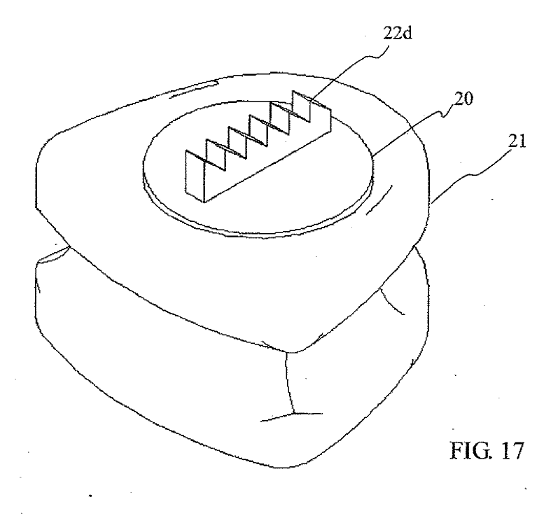

An intervertebral filling constructed according to a seventh preferred embodiment of the present invention is shown in FIG. 17, which has a construction similar to the first embodiments shown in FIGS. 5 and 6 except a major difference that a pair of toothed bars 22d having a plurality of teeth are provided on the two axial ends of the supporting body 20 with the sharp teeth of the toothed bar 22d protruding from the buffer layer 21 to replace the position pins.

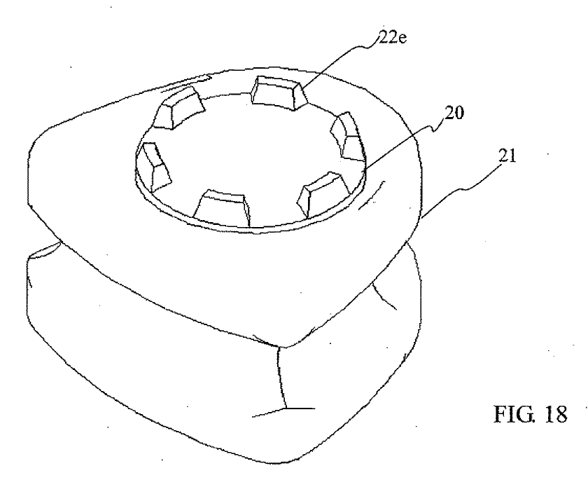

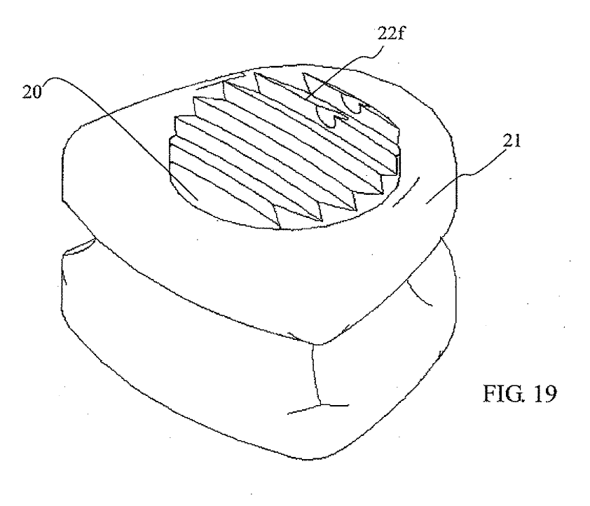

An intervertebral filling constructed according to an eighth preferred embodiment of the present invention is shown in FIG. 18, which has a construction similar to the first embodiments shown in FIGS. 5 and 6 except a major difference that six positioning wedges 22e are provided on each one of the two axial ends of the supporting body 20 with the positioning wedges 22e protruding from the buffer layer 21 to replace the position pins.

An intervertebral filling constructed according to a ninth preferred embodiment of the present invention is shown in FIG. 19, which has a construction similar to the first embodiments shown in FIGS. 5 and 6 except a major difference that each one of the two axial ends of the supporting body 20 is provided with a corrugated surface with the sharp ridges 22f protruding from the buffer layer 21 to replace the position pins.

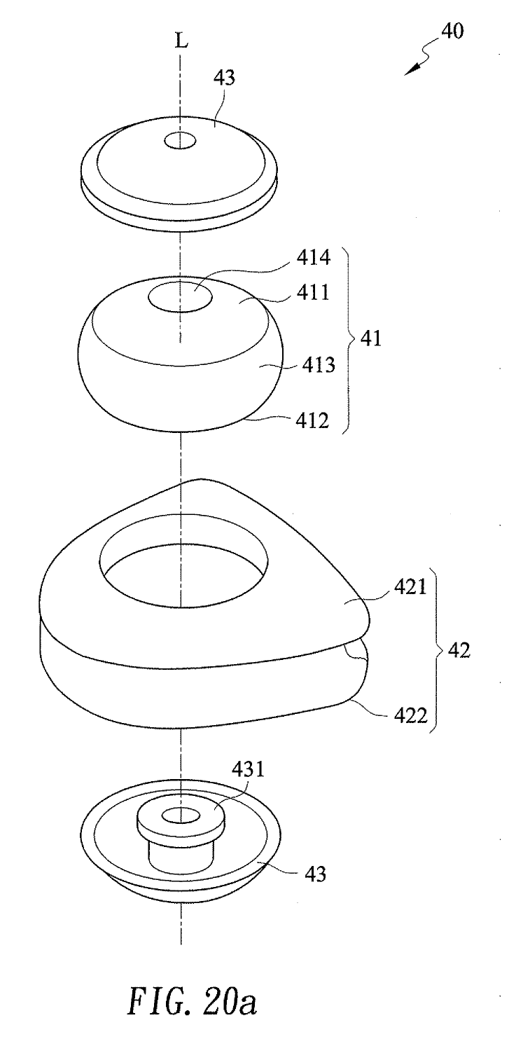

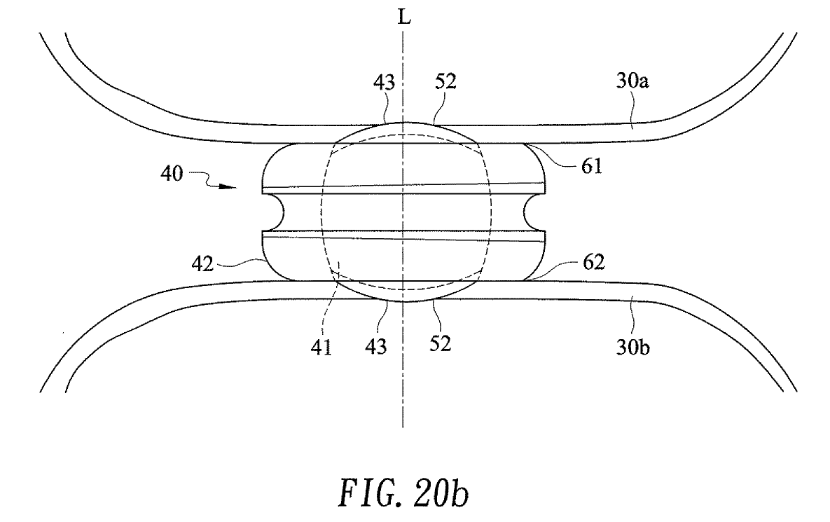

An intervertebral filling 40 constructed according to a tenth preferred embodiment of the present invention is shown in FIG. 20a and FIG. 20b. The intervertebral filling 40 has a cylindrical support part 41 and two caps 43 connected to an upper end 411 and a lower end 412 of the cylindrical support part 41; and a buffer layer 42 surrounding a longitudinal surface 413 of the cylindrical support part 41.

The buffer layer 42 has a concave groove recessed perpendicularly to the direction of a center axis L of the intervertebral filling 40 and on a peripheral surface between an upper surface 421 and a lower surface 422 of the buffer layer 42. The upper surface 421 of the buffer layer 42 and the cap 43 connected to the upper end of the cylindrical support part 41 together form a first surface 61 of the intervertebral filling 40, and the lower surface 422 of the buffer layer 42 and the cap 43 connected to the lower end of the cylindrical support part 41 together form a second surface 62 of the intervertebral filling 40. The intervertebral filling 40 is placed between the two cervical vertebrae end-plates 30a and 30b, so that the first surface and the second surface of the intervertebral filling contact the two cervical vertebrae end-plates 30a and 30b.

Each of the two caps 43 has a post 431, and the cylindrical support part 41 has a hole 414 on the upper end and another hole on the lower end thereof (not shown in the drawings) corresponding to the posts 431, and the two caps 43 are connected to the upper end 411 and the lower end 412 of the cylindrical support part 41 separately with the posts 431 of the two caps 43 being received in said hole 414 and said another hole of the cylindrical support part 41.

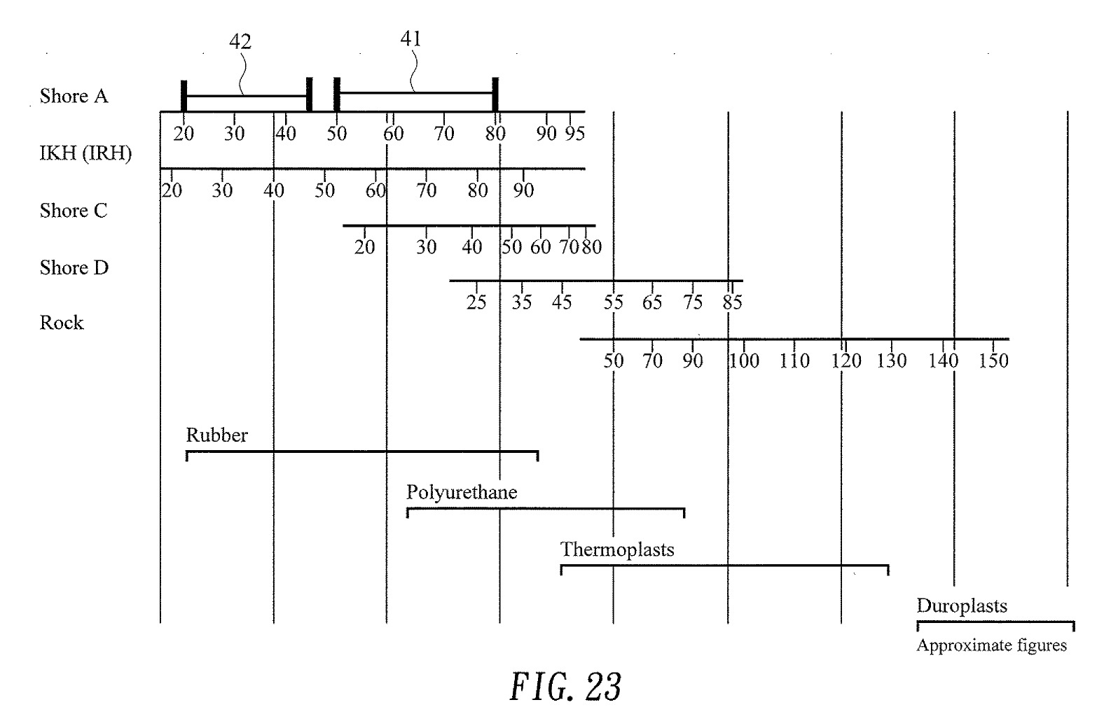

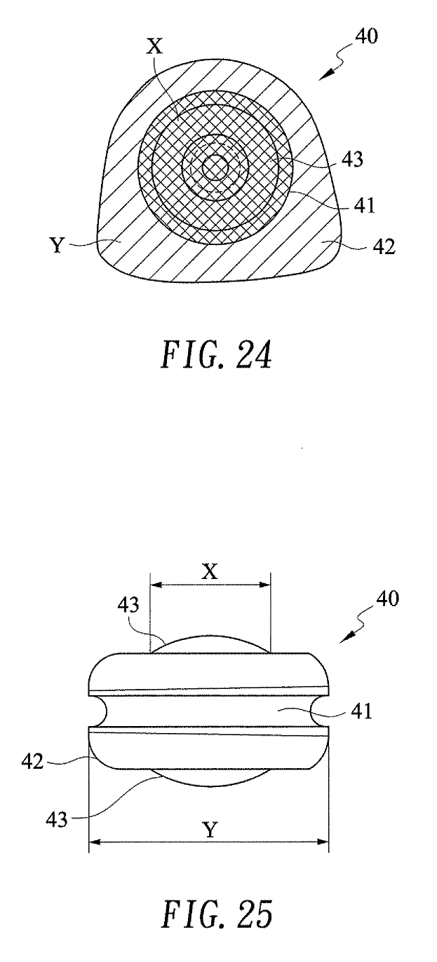

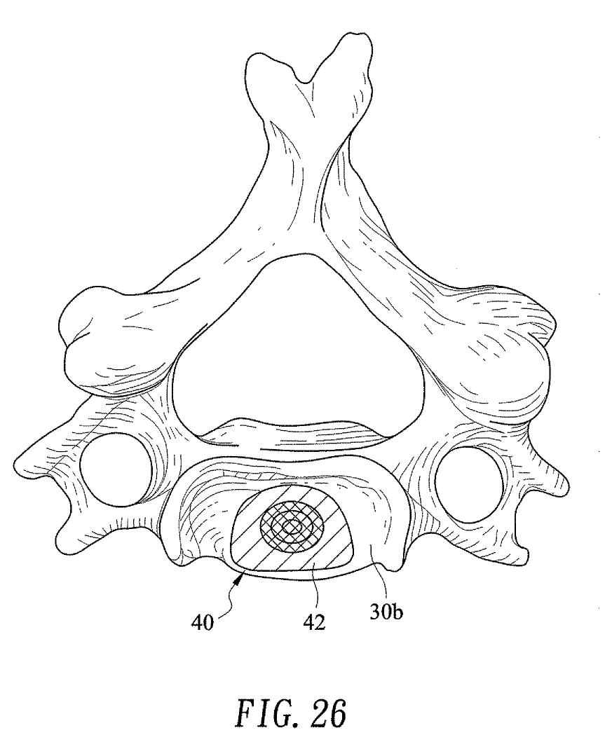

The cylindrical support part 41 has a Shore A hardness of 50-80 and the buffer layer 42 has a Shore A hardness of 20-45, and the cylindrical support part 41 and the buffer layer 42 are independently made of pharmaceutically acceptable polymeric materials as shown in FIG. 23, such as silicone rubber or polyurethane elastomer.

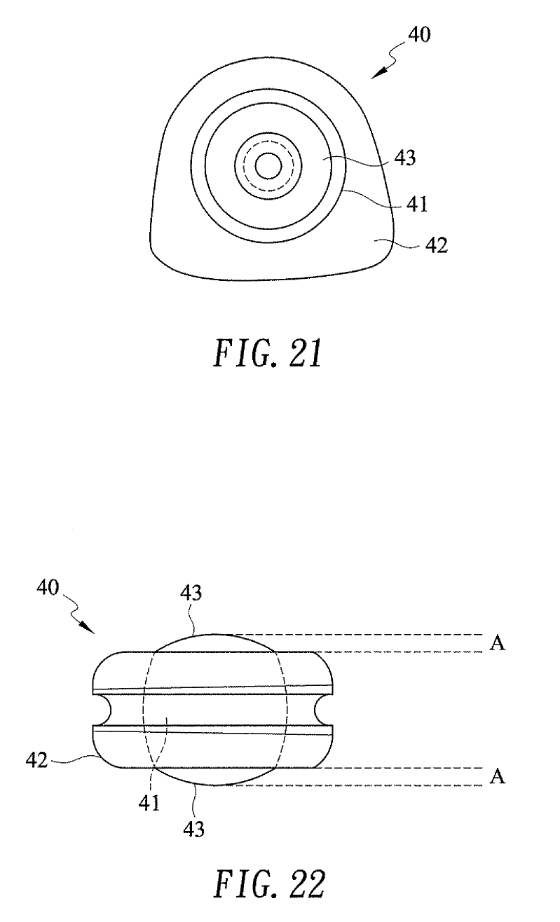

As shown in FIGS. 21 and 22, the intervertebral filling 40 is assembled by suing the cylindrical support part 41; the two caps 43; and the buffer layer 42, so that the buffer layer 42 surrounds and is contiguous with the cylindrical support part 41 with the two caps 43 protruding from the buffer layer 42 with a height A of about 1 mm to about 3 mm, respectively.

As shown in FIG. 20b, the two caps 43 contact the two cervical vertebrae end-plates 30a and 30b, separately, wherein the two caps 43 are received in two corresponding recesses 51 and 52 formed in advance on the two cervical vertebrae end-plates respectively, while the buffer layer 42 is conformably pressed by portions surrounding the recesses 51 and 52 which remain intact on the two cervical vertebrae end-plates 30a and 30b. The recesses 51 and 52 are formed by polishing off only a relatively small portion of the end-plates 30a and 30b in view of the height A (shown in FIG. 22) being only about 1-3 mm. Moreover, the two caps 43 have porous surfaces, which allow bone cell growing in after the intervertebral filling 40 being implanted between two cervical vertebrae end-plates 30a and 30b.

As shown in FIGS. 24, 25 and 26, the intervertebral filling 40 of the present invention has a size and a shape suitable for being implanted between two cervical vertebrae end-plates 30a and 30b, wherein the cap 43 thereof has an exposing area X which corresponds to the area on the end-plate needs to be polished off and also the area with a support from the cylindrical support part 41. The polishing off of the end-plate will enhance the growth of bone cells from the end-plate into the porous surface of the cap 43, and thus enhance the fusion of the intervertebral filling 40 with the end-plates. The buffer layer 42 together with the caps 43 form the first surface and the second surface of the intervertebral filling 40, which have an area Y greater than the area X. The buffer layer 42 is softer than the cylindrical support body 41 and is pressed between the intact portions of the end-plates, which can prevent the intervertebral filling 40 from sinking into and slipping from the end-plates.

Although the present invention has been explained in relation to its preferred embodiment, it is to be understood that many other possible modifications and variations can be made without departing from the spirit and scope of the invention as hereinafter claimed.

What is claimed is:

1. An intervertebral filling for placement between two adjacent cervical vertebrae end-plates in a human body, the intervertebral filling comprising:
a first surface and a second surface wherein the intervertebral filling is capable of being placed between two cervical vertebrae end-plates so that the first surface and the second surface of the intervertebral filling contact the two cervical vertebrae end-plates and provide support between the two cervical vertebrae end-plates;
a supporting body having an upper surface, a lower surface and a longitudinal surface extending from the upper surface to the lower surface in a continuous manner, wherein the supporting body is positioned in the intervertebral filling in an axial position so that the upper surface of the supporting body is a part of the first surface of the intervertebral filling and the lower surface of the supporting body is a part of the second surface of the intervertebral filling; and
a buffer layer which surrounds and is contiguous with the longitudinal surface of the supporting body,
wherein the supporting body comprises a cylindrical support part and two caps connected to an upper end and a lower end of the cylindrical support separately, wherein the buffer layer surrounds and is contiguous with the cylindrical support part so that the two caps protrudes from the buffer layer with a height of about 1 mm to about 3 mm, respectively, whereby the two caps can be received in two corresponding recesses formed on the two cervical vertebrae end-plates respectively, while the buffer layer is conformably pressed by intact portions on the two cervical vertebrae end-plates, wherein the cylindrical support part has a Shore A hardness of 50-80 and the buffer layer has a Shore A hardness of 20-45, and the cylindrical support part and the buffer layer are independently made of pharmaceutically acceptable polymeric materials.

2. The intervertebral filling of claim 1, wherein the cylindrical support part and the buffer layer are independently made of silicone rubber or polyurethane elastomer.

3. The intervertebral filling of claim 1, wherein the two caps are porous metallic caps.

4. The intervertebral filling of claim 1, wherein each of the two caps has a post, and the cylindrical support part has a hole on the upper end and another hole on the lower end thereof corresponding to the posts, and the two caps are connected to the upper end and the lower end of the cylindrical support part separately with the posts of the two caps being received in said hole and said another hole.

5. The intervertebral filling of claim 1, wherein the two caps of the supporting body form the upper surface and the lower surface of the supporting body, and the cylindrical support part forms the longitudinal surface of the supporting body.
